(19) United States
(12) Patent Application Publication
(10) Pub. No.: US 2022/0408729 A1
(43) Pub. Date: Dec. 29, 2022

(54) COMPOSITIONS CONTAINING METALLIC COMPOUNDS FOR PROLONGED DISINFECTION OF SURFACES AND THEIR USES

(71) Applicant: NANOMATERIALES QUIMICOS AVANZADOS, S.A. DE C.V., NUEVO LEON (MX)

(72) Inventors: Guillermo Enrique ACOSTA GONZÁLEZ, Nuevo León (MX); Joel GUTIÉRREZ ANTONIO, Estado de México (MX); Daniel RODRÍGUEZ OROZCO, Nuevo León (MX); Omar TORRES LÓPEZ, Nuevo León (MX)

(21) Appl. No.: 17/755,823

(22) PCT Filed: Oct. 19, 2020
(86) PCT No.: PCT/IB2020/059828

§ 371 Date: May 9, 2022

(51) Int. Cl. A01N 59/20; A01N 59/16; A01N 31/02; A01N 33/12; A01P 1/00; A01N 65/36; A01N 25/34; A61K 8/27; A61K 8/19; A61Q 17/00

(52) U.S. Cl. CPC A01N 59/20; A01N 59/16; A01N 31/02; A01N 33/12; A01P 1/00; A01N 65/36; A01N 25/34; A61K 8/27; A61K 8/19; A61Q 17/005

(57) ABSTRACT

The present invention is related to the field of liquid disinfectant formulas, specifically with obtaining disinfectant compositions applicable to hard and smooth surfaces, porous surfaces and for human use; particularly with disinfectant compositions that allow the surface or surfaces where it is applied, a disinfection with a prolonged effect, for periods greater than 24 hours. More particularly with the use of long-lasting disinfectant compounds, some of them approved by international agencies such as the EPA, and which are also approved by the FDA for cosmetic use due to their low or null toxicity. More particularly, the present invention relates to the formulation of said compositions.

### FIELD OF THE INVENTION

The present invention is related to the formulation of liquid disinfectants, specifically with obtaining compositions for the disinfection of hard and smooth surfaces, porous surfaces, and for human use; particularly with compositions that contain various compounds and metal oxides, providing a prolonged effect on the surface or surfaces where it is applied to obtain disinfection, for periods greater than 24 hours. More particularly with the use of long-lasting, low, or non-toxic disinfectants and approved even for cosmetic use. In another aspect, the present invention is referring to the manufacture of said compositions.

### BACKGROUND OF THE INVENTION

The disinfection of surfaces has become a necessity in today's world, in the diary people have contact with different surfaces such as furniture, textiles, devices, tools, means of transport, etc. Because these surfaces can become contaminated with microorganisms such as bacteria, fungi, and viruses; It is necessary to have tools that ensure the elimination of said microorganisms and the health of surfaces for a long time.

The issue of surface transmission is a worrying issue since it has been pointed out that some microorganisms have a prolonged lifetime on surfaces such as stainless steel, plastic, and cardboard; managing to remain on these surfaces for several days. Such surface stability of microorganisms contributes to disease transmission.

Although there are different personal and surface hygiene and disinfection measures, solutions are required that not only ensure the total elimination of microorganisms from surfaces but also have a long-term residual effect that prevents the reinsertion of the said pathogen. as well as prevention and sanitization tasks.

Metallic compounds have been extensively studied to prevent, for example, the RNA replication of viruses such as Coronavirus, thus forming a viable prevention option for the said virus, in addition to being a low-toxicity compound.

The operation of these compounds is because, when applied to a surface or substrate, they form a layer composed of the metallic compound which prevents the replication of microorganisms.

Additionally, it should be noted that some of these metallic compounds, such as the case of zinc compounds, are validated by US and European agencies as a sanitizer and as a safe chemical even in applications for cosmetic formulations.

Within this context, the application of zinc compounds to provide surfaces with prolonged effect protection against microorganisms is well known, for example, patent EP1991209A2 disclosed the use of metallic nanoparticles such as zinc for prolonged protection against viruses such as SARS and other respiratory viruses in surgical materials. Although this patent describes its effectiveness against various viruses, it is a fixed effect on the surface and does not have the versatility of a sanitizing cleaner. U.S. Pat. No. 5,728,404A protects an alcohol-based disinfectant that uses metallic compounds to improve the compatibility of the disinfectant with skin and surfaces, in addition to improving the antimicrobial action of the disinfectant. However, the range of action or time or conditions of patent protection are not declared.

Patent JP2000288108A protects the use of a mask for medical use made of non-woven fabric treated with metal salts such as titanium or zinc oxide and salts to obtain antimicrobial properties. Similarly, the application of salts is focused on a particular type of surface.

U.S. Pat. No. 6,106,854A protects a long-lasting disinfectant for medical and dental applications composed of hydrogen peroxide, an iodine solution, a disinfectant, and an astringent layer composed of acid and basic salts, an antioxidant agent, and an abrasive agent. Although the declared formula is very complete, its content and the shelf life of compounds such as peroxides are very limited, and it is also focused on the specialized health sector.

Another patent, CN101999409A, describes a silver-zinc complex with highly stable antimicrobial properties, however, said patent does not contemplate a long-term effect on the surfaces on which it is applied, in addition to mentioning the use of reducing agents such as sodium borohydride or peroxides.

Within medical applications, patents such as U.S. Pat. No. 6,379,712B1 disclosed the use of silver nanoparticles with long-lasting antimicrobial and antifungal properties; for its part, patent U.S. Pat. No. 3,930,000A for its part describes the application of an allanthionate formed from silver and zinc to create an ointment to combat the appearance of fungi and bacteria. However, the claimed applications are for pharmacological use, furthermore, that silver is a high-cost compound, lowering its competitiveness for commercial applications.

Although there are multiple patents such as WO2008154395AI which protects a disinfectant for use on skin with extended duration, most of these are based on quaternary salts and cationic surfactants which are or may become reserved in their use by international regulations.

Other patents such as U.S. Pat. No. 9,446,071B2 protect an antimicrobial composition of zinc salts and oxide, monoalkyl glyceryl ether, an antioxidant, and optionally an alkaloid. However, this formula protects a cosmetic formulation to combat microorganisms that cause skin conditions, in addition to not taking advantage of a long-lasting effect. Lastly, patents such as WO2008089822A2 protect an antimicrobial composition of zinc oxide, barium sulfate and silver ions for the formulation of disinfectant products, including cosmetics; however, this once again active composition is expensive, and although it is effective in generating a long-term effect, it does not show its immediate disinfecting effect.

### SUMMARY OF THE INVENTION

The present invention discloses a composition with a double antimicrobial effect: in the first effect it acts as a broad-spectrum disinfectant, eliminating microorganisms immediately (30 seconds); The second effect is observed by leaving a protective layer that includes the metal salts and oxides of the composition, which remain on the surface for long periods of time, protecting the covered area against contamination by new microorganisms.

In addition, the present invention uses low toxicity compounds approved by international standards, even being able to be used for topical or cosmetic use, due to the simplicity and effectiveness of the composition, it allows a wide variety of uses and applications related to disinfection.

The present invention is directed to the field of liquid disinfectant formulas, specifically with obtaining compositions for the disinfection of hard, soft, smooth and porous surfaces, as well as for being in contact with the skin.

Furthermore, the present invention refers to compositions that confer a long-lasting sanitizing effect to the surface or surfaces where they are applied, for periods greater than 24 hours. It should be noted that the present invention describes a composition or compositions for long-lasting sanitizing use, some of which are approved by international agencies such as the EPA, and which are also approved by the FDA for cosmetic use due to their low or null toxicity.

One of the objectives of the composition of the present invention is its content, which is composed of a disinfectant for the immediate elimination of microorganisms, compounds and metal oxides for the creation of a long-lasting sanitizing layer on the surface, a fixing agent and process facilitating compounds, or product appearance.

One of the objectives of the composition of the present invention is the immediate elimination of microorganisms, leaving a long-lasting protective layer, with remanence of elimination from smooth and porous surfaces greater than 24 hours.

Another objective of the present invention is to provide a composition that uses metallic disinfectant compounds in its formula, which are safe to use due to their low toxicity, in addition to being approved by international regulations even for cosmetic use.

Moreover, the present invention is used in liquid compositions of low or high density, such as a composition in the form of a gel for hands or in the form of solutions, for the preparation of other products that incorporate natural essential oils.

### DETAILED DESCRIPTION OF THE INVENTION

The present invention claims antimicrobial compositions with a sanitizing agent and compounds and metal oxides that provide long-lasting disinfectant activity, for example: 24, 48 and 72 hours; for the elimination of microorganisms on hard, porous surfaces and for cosmetic use. Which can be used in concentrated form, diluted or in combination with other agents.

Specifically, the invention uses a disinfecting agent to eliminate microorganisms in a short or immediate time, as well as metal compounds and oxides, and a fixing agent to provide a long-lasting sanitizing effect.

In accordance with the present invention, the described compositions possess prolonged disinfection activity against multiple organisms such as: bacteria, fungi and viruses; leaving a protective layer of metallic compounds, for example zinc oxide, which guarantees sanitization for a long time, thus avoiding contamination or transmission of microorganisms upon contact with treated surfaces.

Furthermore, the present invention is composed of one or more metal salts, where:
metal compounds and oxides are contained in the composition in a w/w concentration of 0.005% to 60% with a micro or nanometric size of formula

MnXy,
a. where M is a metal such as: +zinc (Zn), copper (Cu), silver (Ag), Titanium (Ti), and a non-metal.
b. where X is: Oxygen (O), Nitrogen (N) or Carbon (C), phosphate (PO4), carbonate (CO3), silicate (SiO4 2), sulfate (SO4), nitrate (NO3), nitrite (NO2); including salts such as: Zinc Sulfate (ZnSO4), Zinc Gluconate (C12H22O14Zn), Dipyrithione (C10H8N2O2S2) such as Zinc Pyrithione (C10H8N2O2S2Zn), Copper II Sulfate (CuSO4), Copper I Sulfate (Cu2SO4), Silver Sulfate (Ag2SO4), AgCl, among others; and
c. n is (1-4)
d. y is (1-2)

Likewise, the present invention comprises other disinfectants or quaternary ammonium salts in a w/w content of 0.00001 to 70%, where the other disinfectants or quaternary salts are selected from the group comprising: triclosan, benzalkonium chloride, 2-amino-2-methyl-1-propanol, benzalkonium chloride (C6H5CH2N(CH3)2RC), didecyldimethylammonium chloride, mecetronium ethyl sulfate, octenidine, polyhexamethylene biguanide, chlorhexidine gluconate, chlorhexidine acetate, cetrimide, cetylpyridinium chloride, hexetidine, alkylthiuronium compounds, benzyl alcohol, phenoxyethanol, phenoxypropanols, ethylhexylglycerol, undecylenic acid, ethyl alcohol, isopropyl alcohol, 2-biphenylol, triclosan, p-chloro-m-xylenol, thymol, sodium benzoate, 2-phenoxyethanol, methylisothiazolinone or other quaternary salts, as well as cationic or antimicrobial surfactants, as well as mixtures of any of these.

In accordance with the present invention, the metallic compounds contained in the present invention may be present in a w/w percentage of 0.005% to 60%, being, for example, zinc oxide and zinc pyrithione.

In another aspect of the present invention, the disinfectant agent of the composition is found in a concentration of 0.0001% to 10.0% p/p and can be selected from the following examples: triclosan, 2-amino-2-methyl-1-propanol, Benzalkonium Chloride (C6H5CH2N(CH3)2RC), Didecyldimethylammonium Chloride, Mecetronium Ethyl Sulfate, Octenidine, Polyhexamethylene Biguanide, Chlorhexidine Gluconate, Chlorhexidine Acetate, Cetrimide, Cetylpyridinium Chloride, Hexetidine, Alkylthiuronium Compounds, Benzyl Alcohol, Phenoxyethanol, Phenoxypropanols, ethylhexylglycerol, undecylenic acid, 2-biphenylol, p-chloro-m-xylenol, thymol, sodium benzoate, 2-Phenoxyethanol, methylisothiazolinone, butylisothiazolinone or other quaternary salts, cationic or antimicrobial surfactants, as well as mixtures of any of these, in addition to extracts of natural or citrus oils p/p or alcohol-based disinfectants in a concentration of 20 to 90% v/v.) and can be selected, for example, from ethyl alcohol or isopropyl alcohol, a fixing agent in a concentration of 0.005% to 15% p/p, said fixing agent being one selected from the group comprising Carboxy Methyl Cellulose (CMC), Hydroxyethyl cellulose, methyl ethyl hydroxyethyl cellulose, Xanthan Gum, glyceryl acetate, carbopol, polyvinyl alcohol, polymers such as: polyurethanes, polystyrenes, combinations with latex, polyacrylates, and vinyl, as well as natural fixatives such as: nopal mucilage, flaxseed mucilage, chia mucilage, pitch, resins, among others. others, and the combination thereof, and the rest of the components in a concentration of 0.001 to 20% p/p.

In another aspect, the composition of the present invention can be used in the preparation of a composition in the form of a hand sanitizing gel, for which it would be used in a concentration of 0.001 to 2% p/p; adding alcohol in a concentration of 30 to 80% v/v, said alcohol being, for example, ethyl alcohol; a thickening agent in a concentration of 0.01 to 25% w/w, said thickening agent being, for example, carbopol and triethanolamine; moisturizing or emollient agents and fragrance.

The vehicle used in all the applications of the present composition is deionized water, in a percentage of 30% to 99.5% p/p until completing 100% of the weight of the composition.

Additionally, the invention may contain some essential oils to provide aroma and help as natural complements for disinfection, such as, for example, orange or citrus oil terpenes in a percentage of 0.005% to 5% p/p.

The combination of the ingredients described above allows the manufacture of disinfectant compositions that have this immediate activity and prolonged sanitizing action. The disinfectant activity of the present invention is efficient in the elimination of pathogenic microorganisms, such as:
gram+ and gram− bacteria such as Escherichia coli, Staphylococcus aureus, Vibrio cholerae, Salmonella enterica, Pseudomonas aeruginosa, among others;
fungi such as Candida albicans, Penicillium chrysogenum, Aspergillus, Trichophyton rubrum, Rhizopus nigricans, Alternaria alternata, Mucor corymbilfer, Mucor mucedo, Saccharomyces cerevisiae, Schizosaccharomyces pombe, Cryptococcus neoformans; and
viruses with and without enteric coating such as Influenza A(H1N1), SARS-CoV-2, Influenza A(H3N2), Influenza A(H7N9), Rhinovirus, Rotavirus, RSV, Hepatitis A, Adenovirus, Coronavirus, among others.

respectively, preserving its antimicrobial effect for at least 24 hours and up to 120 hours later.

The compositions of the claimed invention have been tested on hard and smooth surfaces in accordance with standard tests such as the ISO 22196 method in which an inoculum of known concentration of the microorganism under test is placed on the surface modified or previously treated with the product of the present invention and is incubated under controlled conditions for 24 hours, to prevent its evaporation, favoring microbial development, after which time the inoculum is extracted from the tested surface and a reference control sample without antimicrobial activity, the results are expressed in percentage or logarithm base 10 of the reduction of the microbial population of the sample against the reference control, in another test the determination of the minimum inhibitory and bactericidal concentration of the product of the present invention in aqueous solutions is carried out by the M100 method of the CLSI in which test tubes with different concentrates are prepared tions of the germicidal product of the present invention with culture medium and physiological solution, adding a known quantity of microorganisms, the result is expressed in concentration units that are equivalent to that where the cut-off point between microbial growth and inhibition occurs. or biocidal effect where appropriate; In another evaluation, the sample is placed with 1% of a suspension of the inoculum with the microorganism in a concentration close to 1×1010 CFU/ml and is sampled at times of 30 seconds and up to 20 minutes to determine the efficiency of the elimination of microorganisms. in a given contact time, the results are commonly expressed in % removal based on NMX-BB-040-SCFI-1999; In another test method, a culture medium is prepared in agar previously inoculated with the microorganism of interest at a rate of 103-104 CFU/ml. Once the agar solidifies, it is incubated for 24 to 48 hours in contact with the treated surface. with the product of the present invention, the results are observed in the distance of the inhibition halo that is generated by the elimination-inhibition effect of microbial growth, this test is recognized as the Kirby Bauer method.

It is important to point out that the composition of the present invention also demonstrates its effectiveness on porous surfaces, for example, surgical-type fabric such as non-woven fabrics such as face masks, gowns, medical overalls, and caps.

The present invention may additionally include processing aids or appearance aids in order to improve its appearance for the consumer, such as aroma, stabilizers, water hardness aids, preservatives, thickeners, colorants, among others. This allows generating compositions with the same properties as those previously described. The selection of any of these components is based on aesthetic and/or functional considerations.

Additionally, the present invention can be used in its concentrated or diluted form to generate hand sanitizing gels such as the one described above, surface cleaners, solutions for fabric treatments, solutions for industrial disinfection through mechanical or pneumatic applicators, solutions for disinfectant mats, multipurpose disinfectants for the home, multipurpose disinfectants for clothes, among other applications.

The present invention can be used in its concentrated or diluted form up to a ratio of vehicle:concentrate 10,000:1 p/p, for prolonged disinfection of porous surfaces such as natural, synthetic, woven and non-woven fabrics, cosmetic use, as well as smooth solid surfaces; such as polymers, ceramics, steel, glass, wood, vinyl coatings, paints, enamels, among others.

### EXAMPLES

The examples that are included below are for the sole purpose of illustrating the present invention, without implying any limitation to its scope:

### Example 1. Performance Tests of the Composition of the Invention in Dilution

By testing the present invention at a 1000:1 dilution, and subjecting it to a microbial challenge assay in accordance with NMX-BB-040-SCFI-1999 with a contact time of 30 seconds, using Staphylococcus aureus as the test microorganism, and an inoculum concentration of 75-125 x108 CFU/mL, a microorganism elimination of 99.99% is obtained.

### Example 2. Test on Fabrics at a Prolonged Time of 24 Hours

The same previously described dilution of the invention 1000:1 p/p was tested, evaluating its biocidal performance when applied by spraying on different fabrics for medical use such as blue disposable face mask, surgical gown, medical coverall (isolation suit), disposable cap.

A sample was taken from each of these fabrics, the diluted product was applied by spray on the outside of the fabric and allowed to dry, the samples were allowed to dry and rinsed in deionized water to be evaluated 24 hours later. Finally, for the Kirby Bauer test, a 1×1 cm sample was cut from each fabric.

The inhibition halo was determined in each of the treated fabrics, finding inhibition halos that are representative of the germicidal effect of the surface.

### Example 3. Application of the Invention in a Sanitizing Gel Formulation

The present invention was used in a sanitizing gel in a ratio close to 10,000:1, using: ethanol, a humectant, a polymeric thickener, fragrance, and water as part of the gel formulation. Said composition was evaluated according to NMX-BB-040-SCFI-1999 with a contact time of 30 seconds, using Staphylococcus aureus and Escherichia coli as test microorganisms, and an inoculum concentration of 75-125 x108 CFU/mL; obtaining, as a result, the elimination of 99.99% of said microorganisms and verifying the effectiveness of the invention.

### Example 4. Application of the Invention in the Formulation of Antibacterial and Antifungal Paints

The present invention was used as an additive in a water-based matte vinyl paint formulated without the presence of any biocide or fungicide, this paint formulation was divided into two portions, one as a reference and the other with the addition of 0.1% to 0.5% of the formulation of the present invention, its effect for the inhibition of the proliferation of bacteria, yeasts and fungi in packaging and in dry film was evaluated by ISO 22196: 2011 using Staphylococcus aureus, Escherichia coli, Aspergillus oryzae and Candida albicans as microorganisms of test, obtaining as a result the elimination of 99.9% of said microorganisms against the reference sample at different evaluation times (24 hours, 7 days, 14 days, 28 days) of samples subjected to accelerated weathering conditions in a UV light chamber at 330 nm with periods of 20 hours of light and 4 hours of darkness per day; For the antimicrobial evaluation tests in the container, inoculation tests were carried out with a medium contaminated with a concentration of 1.5×106 CFU/mL of the fungus Aspergillus oryzae and the same concentration of the yeast Pichia membranifaciens in three times, verifying the viability of the microorganisms after 72 hours of inoculation, finding >10 CFU/mL in the container with the additive of the present invention against uncountable concentrations in the reference sample, verifying the effectiveness of the invention.

### Example 5. SARS-COV2 Virus Elimination Test of the Composition of the Present Invention is Diluted

When testing the composition of the present invention in a 0.1% aqueous solution, in the presence of SARS-CoV-2 virus, in a ratio of 1:9 p/p (one part of the suspension with viral particles and 9 parts of the composition). Where the initial viral concentration was 1000 viral particles per microliter. After 10 minutes of contact, the viral genetic material could not be detected by RT-PCR, which represents a total elimination of the virus. Evaluations were made by placing the formulation of the present invention at 10% w/w in an aqueous medium in the presence of the virus, 1 part of virus per 9 of the composition of the invention; the initial concentration was 11,000 virus particles per microliter, after 10 minutes of contact, and after genetic analysis by RT-PCR, it was observed that the formulation completely destroyed the viral genetic material, since, like the positive control, viral RNA was not detected, which was not the case in the negative control, where the presence of the viral genome was observed. The analysis was repeated by placing the formulation of the present invention at 1% w/w in the presence of the virus, in a w/w ratio of 1:9, after 20 min of contact, the viral genetic material was completely destroyed.

### Example 6. Antimicrobial Efficacy of Products Formulated with the Composition of the Present Invention

Tests were carried out with the formulations of moisturizing creams, tonics, sanitizers, soap, shampoo, and cosmetics such as sunscreens formulated with 0.05 to 2% of the composition of the present invention evaluated by the Kirby Bauer method and microbial challenge which showed halos of inhibition of 1 to 6 mm and values of 99.99% inhibition in times of the 30 seconds to 10 minutes against Staphylococcus aureus and Escherichia coli.

### Example 7. Antimicrobial Efficacy of Surfaces Created with the Inclusion of the Additive of the Present Invention and with Coatings Containing the Composition of the Present Invention

Samples of tanned leather, shoe soles, epoxy coatings and plastics such as high-density polyethylene, low-density polyethylene, PVC, EVA, polypropylene, added with the formulation of the present invention in concentrations of 0.2 to 2% evaluated by means of the ISO 22196:2011 standard with results of inhibition of Staphylococcus aureus and Escherichia coli from 95 to 99.99%

### Example 8 Validation of the Residual Effect of the Product of the Present Invention

The product of the present invention was applied to the various surfaces of a double-section articulated urban bus, applying the product to windows, seats, handrails, dashboard, the steering wheel, articulated axle and handles, allowing it to dry to fix and form a layer constant and prolonged effect sanitizer, which is evaluated in the field by taking samples with a flexible culture plate (Petri film) for total aerobes, the results of the counts were compared with an identical bus on the same route with an influx of 6000 users per day, the comparative results of the sampling carried out on 3 consecutive days with samples taken at the beginning of the day, in the middle of the day and at the end of the day, show a decrease in the incidence of counts of microorganisms of 50% of reduction compared to the control bus even under normal use conditions without the need for re-application during the day.

The examples and embodiments described herein are for interpretation purposes only, and various improvements or modifications in view thereof would be suggested to those skilled in the art and are within the spirit and scope of the present application and the scope of the appended claims.

1. Disinfectant composition comprising:
one or several metallic salts that act as a long-lasting sanitizing agent of a micro or nanometric size, in a concentration p/p of 0.005% to 60%, the metallic salts having a formula:
MnXy,
to. where M is a metal such as: +zinc (Zn), copper (Cu), silver (Ag), Titanium (Ti);
b. where X is a non-metal such as: Oxygen (O), Nitrogen (N) or Carbon (C), phosphate (PO4), carbonate (CO3), silicate (SiO4 2), sulfate (SO4), nitrate (NO3), nitrite (NO2); including salts such as: Zinc Gluconate (C12H22O14Zn), dipyrithione (C10H8N2O2S2) such as Zinc Pyrithione (C10H8N2O2S2Zn), among others; and where
c. n (1-4)
d. y (1-2)
a disinfectant such as ethanol, isopropanol or quaternary ammonium salts in content w/w from 0.00001 to 70%, which shows a broad spectrum, eliminating microorganisms in a short period of time such as 30 seconds;
a fixing agent in a concentration of 0.005% to 15% w/w; and
Compounds that facilitate the process or the appearance of the product.

2. Disinfectant composition according to claim 1, characterized in that it comprises a prolonged disinfection activity against multiple organisms such as:
gram+ and gram− bacteria such as: Escherichia coli, Staphylococcus aureus, Vibrio cholerae, Salmonella enterica, Pseudomonas aeruginosa, among others;
fungi such as: Candida albicans, Penicillium chrysogenum, Aspergillus, Trichophyton rubrum, Rhizopus nigricans, Alternaria alternata, Mucor corymbilfer, Mucor mucedo, Saccharomyces cerevisiae, Schizosaccharomyces pombe, Cryptococcus neoformans; and
viruses with and without enteric coating such as: Influenza A(H1N1), SARS-CoV-2, Influenza A(H3N2), Influenza A(H7N9), Rhinovirus, Rotavirus, RSV, Hepatitis A, Adenovirus, Coronavirus, among others.

3. Disinfectant composition according to claim 1, characterized in that its disinfectant activity is from 24 to 120 hours, then forming a long-lasting disinfectant layer on the surface.

4. Disinfectant composition according to claim 2, characterized in that it forms a protective layer of metallic compounds such as zinc oxide, which guarantees sanitization for a long time.

5. Disinfectant composition according to claim 1, characterized in that it additionally contains some essential oils as natural complements for disinfection, such as orange or citrus oil terpenes in a percentage of 0.005% to 5% p/p

6. Disinfectant composition according to claim 1, characterized in that it additionally comprises other disinfectants or quaternary ammonium salts in a concentration of 0.00001 to 70% w/w, where said other disinfectants or quaternary salts are selected from the group comprising: triclosan, benzalkonium chloride, 2-amino-2-methyl-1-propanol, benzalkonium chloride (C6H5CH2N(CH3)2RC), didecyldimethylammonium chloride, mecetronium ethyl sulfate, octenidine, polyhexamethylene biguanide, chlorhexidine gluconate, chlorhexidine acetate, cetrimide, cetylpyridinium chloride, hexetidine, alkylthiuronium compounds, benzyl alcohol, phenoxyethanol, phenoxypropanols, ethylhexylglycerol, undecylenic acid, 2-biphenylol, triclosan, p-chloro-m-xylenol, thymol, sodium benzoate, 2-Phenoxyethanol, methylisothiazolinone or other quaternary salts, as well as surfactants cationic or antimicrobial, as well as mixtures thereof.

7. Disinfectant composition according to claim 1, characterized in that the metallic disinfectant compounds have low toxicity.

8. Disinfectant composition according to claim 1, characterized in that it also comprises low- or high-density liquid compositions such as compositions in the form of gels for hands or in the form of solutions.

9. Disinfectant composition according to claim 8, characterized in that it also contains extracts of natural or citrus oils, and alcohol-based disinfectants in a concentration of 20 to 90% v/v, such as ethyl alcohol or isopropyl alcohol.

10. Disinfectant composition according to claim 1, characterized in that the fixing agent is selected from the group of:
Carboxy Methyl Cellulose (CMC), Hydroxyethyl Cellulose, Methyl Ethyl Hydroxyethyl Cellulose, Xanthan Gum, Glyceryl Acetate, Carbopol, Polyvinyl Alcohol,
polymers such as: polyurethanes, polystyrenes, combinations with latex, polyacrylates, and vinyl,
natural fixatives such as: nopal mucilage, flaxseed mucilage, chia mucilage, pitch, resins, among others, and their combination.

11. Disinfectant composition according to claim 1, characterized in that said composition is effective on porous surfaces, for example, surgical-type fabric such as: non-woven fabrics such as face masks, gowns, medical overalls, and caps.

12. Disinfectant composition according to claim 1, characterized in that it is formulated as hand sanitizing gels, surface cleaners, solutions for fabric treatments, solutions for industrial disinfection through mechanical or pneumatic applicators, solutions for disinfectant mats, multipurpose disinfectants for the home, multipurpose disinfectants for clothing, natural, synthetic, woven and non-woven fabrics, for cosmetic use, as well as on smooth solid surfaces; such as: polymers, ceramics, steel, glass, wood, vinyl coatings, paints, enamels.

13. Disinfectant composition according to claim 12, characterized in that it is present in a proportion of vehicle:concentrate 10,000:1 p/p.

14. Disinfectant composition according to claim 1, characterized in that it is in the form of a sanitizing gel with a concentration of 0.001 to 2% p/p; what includes
alcohol in a concentration of 30 to 80% v/v, said alcohol being for example ethyl alcohol;
a thickening agent in a concentration of 0.01 to 25% w/w, said thickening agent being for example carbopol and triethanol amine;
moisturizing or emollient agents and fragrance.

15. Disinfectant composition according to claim 1, characterized in that it also includes a vehicle, such as deionized water, in a percentage of 30% to 99.5% p/p or until completing 100% of the weight of the composition.

16. The disinfectant composition according to claim 1, characterized in that it also comprises process or appearance auxiliary substances such as:
thickening substances in a concentration of 0.001 to 25%,
preservatives in a concentration of 0.001 to 15%;
humectants in a concentration of 0.001 to 20%;
dyes in a concentration of 0.001 to 10%;
fragrance in a concentration of 0.001 to 10%;
water softeners in a concentration of 0.001 to 10%;
pH regulators in a concentration of 0.001 to 10%; and
the combination of them, to form a ready-to-use version.

17. The disinfectant composition according to claim 16, characterized in that it also contains a thickening agent in a concentration of 0.01 to 15%, said thickening agent being selected from the group comprising carbopol and triethanol amine.

18. The disinfectant composition according to claim 1, characterized in that it also comprises a concentration of surfactants from 0.1 to 60%.

19. The disinfectant composition according to claim 18, characterized in that the surfactants are selected from: ethoxylated alcohols, sodium lauryl sulfate, sodium lauryl ether sulfate, dodecylbenzenesulfonic acid or its salt, gluconates, sarcosinates, among others to form cleansing formulas.

20. The disinfectant composition according to claim 1, characterized in that it also comprises terpene oils and/or essential oils: rosemary (Salvia rosmarinus), lavender (Lavandula), mint (Mentha), orange (Citrus sinensis), Bergamot (Citrus bergamia), oregano (Origanum vulgare), eucalyptus (Eucalyptus), thyme (Thymus vulgare), pine (Pinus ponderosa), citronella (Citronella), camphor, d-limonene, α-Pinene, β-pinene, eucalyptol (1, 8-cineol), linalool, carvacrol, menthol, cymene, thymol, geraniol, o-cymene, α-turmerone, β-turmerone, piperitone, garlicin.

21. The disinfectant composition according to claim 1, characterized in that it eliminates microorganisms with a contact time of 30 seconds to 15 minutes with a long-lasting antimicrobial effect of 12, 24, 48, 72 hours.

22. The disinfectant composition according to claim 1, characterized in that said composition acts on porous surfaces, such as surgical-type fabrics such as: non-woven fabrics such as face masks, gowns, medical overalls and caps, fields and others.

23. A water-based paint characterized in that it comprises a disinfectant composition as claimed in claim 1 in a range of 0.05% to 3%.

24. The paint according to claim 23, characterized in that the concentration of the disinfectant formulation used inhibits the proliferation of bacteria, yeasts, and fungi.

25. A fabric for medical use characterized in that it has embedded the disinfectant composition claimed in claim 1 in a dilution of 1000:1 p/p.

26. The fabric for medical use according to claim 25, characterized in that it is used in the following products: disposable face mask, surgical gown, medical overalls (isolation suit), disposable cap, medical fields, gloves, shoes disposable among others.

27. A sanitizing formulation characterized in that it includes the disinfectant composition as claimed in claim 1, in an aqueous solution in a 0.1% proportion that contains at least one thickener in a proportion of 0.01 to 10% w/w.

28. A sanitizing formulation according to claim 27, characterized in that its antiviral action is effective at a concentration of 0.1% p/p.

29. A sanitizing formulation according to claim 28, characterized in that the antiviral action also includes SARS-CoV-2.

30. The use of the sanitizing formulation of claim 1, at a concentration from 0.05% to 2% in the preparation of moisturizing creams, tonics, sanitizers, soap, shampoo and cosmetics as protectors solar.

31. The use of the disinfectant composition as claimed in claim 1 for the preparation of a formulation to eliminate microorganisms from any surface, such as:
gram+ and gram− bacteria such as: Escherichia coli, Staphylococcus aureus, Vibrio cholerae, Salmonella enterica, Pseudomonas aeruginosa, among others;
fungi such as: Candida albicans, Penicillium chrysogenum, Aspergillus, Trichophyton rubrum, Rhizopus nigricans, Alternaria alternata, Mucor corymbilfer, Mucor mucedo, Saccharomyces cerevisiae, Schizosaccharomyces pombe, Cryptococcus neoformans; and
viruses with and without enteric coating such as: Influenza A(H1N1), SARS-CoV-2, Influenza A(H3N2), Influenza A(H7N9), Rhinovirus, Rotavirus, RSV, Hepatitis A, Adenovirus, Coronavirus, among others.
